(19) United States
(12) Patent Application Publication
(10) Pub. No.: US 2026/0327315 A1
(43) Pub. Date: Oct. 22, 2026

(54) INNATE LYMPHOID CELLS WITH CHIMERIC ANTIGEN RECEPTORS

(71) Applicant: UNIVERSITY HEALTH NETWORK, Toronto (CA)

(72) Inventors: Sarah CROME, Toronto (CA); Siavash MASHHOURI, Toronto (CA)

(21) Appl. No.: 19/184,913

(22) Filed: Apr. 21, 2025

(51) Int. Cl. C07K 16/28; A61K 40/10; A61K 40/31; A61K 40/41; A61K 40/42; A61P 35/00; A61P 37/06; C07K 14/715; C12N 5/078; C12N 15/86

(52) U.S. Cl. CPC C07K 16/2833; A61K 40/10; A61K 40/31; A61K 40/416; A61K 40/418; A61K 40/4213; A61P 35/00; A61P 37/06; C07K 14/7155; C12N 5/0634; C12N 15/86; C07K 2319/02; C12N 2510/00; C12N 2740/15043

(57) ABSTRACT

There is described herein an innate lymphoid cell (ILC) expressing a chimeric antigen receptor (CAR), the CAR comprising an antigen recognition domain for recognizing a target organ or target cell, a transmembrane domain, and a signalling domain for activating signalling within the ILC.

### FIELD OF THE INVENTION

This application relates to chimeric antigen receptors, including chimeric antigen receptors expressed on innate lymphoid cells.

### BACKGROUND OF THE INVENTION

Regulatory cell therapy is a promising field aimed at restoring immune homeostasis by utilizing cells with immunosuppressive functions. These therapies primarily target autoimmune diseases, transplant rejection, and inflammatory disorders by controlling excessive immune responses and promoting immune tolerance. The main goal is to replace or complement traditional immunosuppressive drugs, which often have broad effects and significant side effects. The most advanced regulatory cell therapy to date involves Tregs, with several clinical trials assessing their efficacy and safety¹. While Tregs hold significant therapeutic promise, their clinical efficacy is often hindered by challenges such as limited numbers, reduced stability, and lack of antigen specificity². However, the limitations associated with Tregs have led to the development of engineered regulatory cells. Chimeric antigen receptor (CAR)-Tregs have emerged as a more refined approach to overcome these limitations³. CAR-Tregs are genetically engineered to express chimeric antigen receptors that recognize specific antigens, thereby allowing them to exert their immunosuppressive effects in a targeted manner. Compared to non-engineered Tregs, CAR-Tregs offer enhanced specificity, improved stability, and greater potency, reducing systemic immunosuppression and lowering the risk of unwanted immune suppression in non-target tissues⁴⁻⁹. Several CAR-Treg therapies are currently in early-phase clinical trials, demonstrating promising results in preclinical models¹⁰.

Innate lymphoid cells (ILCs) have emerged as a promising new class of immune cells with potential applications in cell therapy¹¹. Among them, Type 2 innate lymphoid cells (ILC2s) are of particular interest due to their role in tissue homeostasis and immune regulation. In the context of cancer, ILC2s have been shown to be able to directly kill tumor cells and cell therapy with these cytotoxic ILC2s protected cancer progression³⁴. IL-10-producing ILC2s, on the other hand, have significant potential as a novel regulatory cell therapy¹²⁻¹⁸. By modulating the activity of effector immune cells, IL-10-producing ILC2s could be harnessed in cell therapies to treat inflammatory and autoimmune diseases, as well as to promote tolerance in transplantation. Their natural ability to function in inflamed tissues without requiring antigen-specific activation gives them an advantage over traditional Tregs, which depend on TCR signaling for activation. Additionally, IL-10-producing ILC2s can become tissue-resident and long-lived, providing a sustained immunoregulatory effect without the need for repeated infusions.

### SUMMARY OF THE INVENTION

In an aspect, there is provided an innate lymphoid cell (ILC) expressing a chimeric antigen receptor (CAR), the CAR comprising an antigen recognition domain for recognizing a target organ or target cell, a transmembrane domain, and a signalling domain for activating signalling within the ILC.

In an aspect, there is provided the CAR-expressing ILC described herein for use in the treatment or prevention of, or in the manufacture of a medicament for use in the treatment or prevention of, cancer, an autoimmune disease, transplant rejection, Graft-versus-Host disease, an immunodeficiency disease or an inflammatory disease.

In an aspect, there is provided a method of treating or preventing in a subject, cancer, an autoimmune disease, graft-versus-host disease, an immunodeficiency disease or an inflammatory disease, the method comprising administering to the subject the CAR-expressing ILC described herein.

In an aspect, there is provided a chimeric antigen receptor as described herein.

In an aspect, there is provided a chimeric antigen receptor (CAR), the CAR comprising an antigen recognition domain for recognizing a target organ or target cell, a transmembrane domain, and an ST2 signalling domain.

In an aspect, there is provided a nucleic acid encoding the chimeric antigen receptor described herein.

In an aspect, there is provided a vector comprising the nucleic acid described herein. Preferably, the vector is a viral vector.

In an aspect, there is provided a cell expressing any of the CAR described herein, wherein the cell is a T cell, regulatory T cell (Treg), a myeloid cell or a macrophage, a dendritic cell (DC), a Natural Killer (NK) cell or a B cell.

In a further aspect, there is provided a pharmaceutical composition comprising the CAR expressing cells described herein along with a pharmaceutically acceptable carrier.

### BRIEF DESCRIPTION OF FIGURES

These and other features of the preferred embodiments of the invention will become more apparent in the following detailed description in which reference is made to the appended drawings wherein:

FIG. 1. Generating human CAR-ILC2 (A) Schematic overview of the experimental workflow. Human peripheral blood mononuclear cells (PBMCs) were isolated from healthy donors and enriched for lineage-negative cells. These cells were stained with antibodies to isolate ILC2s, which were then expanded in vitro for three weeks. The expanded ILC2s were transduced with lentiviral particles carrying various CAR constructs. One-week post-transduction, NGFR+ ILC2s were sorted based on surface NGFR expression and further expanded for subsequent in vitro and in vivo analyses. (B) Schematic representation of the anti-HLA-A2 CAR construct alongside a comparative table of different CAR constructs. (C) Illustration depicting the proposed interaction between CAR-ILC2s and pathogenic allogeneic T cells. (D) Dot plots showing successful transduction of human ILC2s with the CAR construct. (E) Cumulative data quantifying transduction efficiency in human ILC2s. (F) Dot plot and histogram illustrating CAR expression stability over 60 days. (G) Dot plots showing cell proliferation dye dilution in transduced (NGFR+) and untransduced (NGFR−) ILC2s after 72-hour co-culture with HLA-A2-expressing K562 cells. (H) Cumulative data comparing cell proliferation dye dilution across different groups. (I) Histogram displaying proliferation of transduced ILC2s under various conditions. (J) Cumulative data showing geometric mean fluorescence intensity (gMFI) of CPD after co-culture with target cells.

FIG. 2: CAR signaling activates ILC2s. (A-B) Histograms and cumulative Log₂ fold change (Log₂FC) of geometric mean fluorescence intensity (gMFI) comparing surface expression of activation markers in different CAR-ILC2s following activation with K562 cells. (C) Log₂FC of cytokine secretion by CAR-ILC2s after activation with K562 cells. (D) Schematic representation of the xenoGVHD animal model. (E) Survival plot of xenoGVHD mice receiving different CAR-ILC2s and non-engineered ILC2s (UT=Untransduced). CD28-CAR (CAR_{CD28z}) ILC2s provide robust protection from GVHD, above that observed with nonengineered (untransduced, UT) ILC2s, CD3-CAR (CAR_{z}) ILC2s, GITR-CAR (CAR_{GITRz}) ILC2s or ICOS-CAR (_{ICOSz}) ILC2s.

FIG. 3: CAR_{CD28z}-ILC2s have enhanced ability to protect from GVHD. CD28 (CAR_{CD28z}) denoted in blue, consistently provide robust protection from GVHD, above that observed with non-engineered (Untransduced, UT) ILC2s (grey) or CD3-CAR (CAR_{z}) (orange). Here, in an independent experiment we see overall survival is ~80% with CD28-CAR-ILC2s, compared to 35-40% with UT or CD3-CAR ILC2s.

FIG. 4: CAR with ST2 domains activates ILC2s. (A) Histogram displaying proliferation of transduced ILC2s under various conditions. (B-C) Histograms and cumulative Log₂ fold change (Log₂FC) of geometric mean fluorescence intensity (gMFI) comparing surface expression of activation markers in different CAR-ILC2s following activation with K562 cells. (D) Cytokine secretion by CAR-ILC2s after activation with K562 cells. (E) CD28 (CAR_{CD28z})-and ST2-CARs (CAR_{ST2})both provide robust protection from GVHD, above that observed with nonengineered (Untransduced, UT) ILC2s, or CD3-CAR (CAR_{z}).

FIG. 5. Human peripheral blood ILC2 highly express CD28 and CD3ζ. (A) Schematic overview of the experimental workflow for CITE-seq of human ILCs. Human PBMCs were isolated from healthy blood and enriched for lineage negative cells. Lineage negative cells were stained with TotalSeq™ C antibodies and submitted for single cell 5′ sequencing by 10× Genomics. To enrich ILCs populations, lineage-depleted cells were purified by FACS sorting and expanded in appropriate media for further analysis. (B) UMAP visualization of all cells (n=50124) color-coded. Clusters were defined by integration-based analysis of all samples and total of 11 clusters were identified. (C) Bar plots illustrating the percentages of each annotated cluster across individual donors. (D-F) Dotplot demonstrating expression of selected and previously described immune subset-specific genes, transcription factors and cell surface markers used to annotate clusters. (G) Violin plots depicting common ILC2 markers, including CD127 (IL7R), CRTH2 (PTGFR2), CD117 (KIT), CD161 (KLRB1), ICOS (ICOS), and TSLPR (IL17RB), across annotated clusters. (H) Dot plots displaying expression levels of selected components of the upstream TCR signaling pathway at both RNA and protein levels.

FIG. 6. Confirmation of expression of CD28 and CD3z in human ILC populations. Gating Strategy for the Identification of total ILC2s, ILC3s/ps and NK subsets from ex vivo PBMCs. Dead cells were excluded, and gating was performed on Lin⁻ and CD3⁻ populations. CD56⁻ and CD16⁻ cells were then selected and further gated on CD127⁺ cells. Within this population, ILC2s were identified as CRTH2⁺ and CD117⁺/⁻, while ILC3s were gated as CD117⁺. Double-negative (DN) cells were classified as ILC1s. (A) Flow cytometry dot plot demonstrating co-expression of CD3ζ and CD28 on ILC1, ILC2, ILC3/p, T cells, CD56 Bright NK and CD56 Dim NK cells. (B) Cumulative data representing both single and co-expression of CD3ζ and CD28 in different immune subsets.

FIG. 7. Human peripheral blood ILC2 express GITR and ICOS, in addition to CD28 and CD3z. (A) Heatmap and dotplot visualization of RNA-level expression of CD3e, CD3z, CD28, ICOS and GITR in all cells (n=50124) across different immune populations. (B) Heatmap and dotplot visualization of protein-level expression of CD3, CD28, ICOS and GITR in all cells across different immune populations.

FIG. 8. (A) Representative Flow cytometry dot plot demonstrating co-expression of CRTH2 and CD28, GITR, ICOS or CD3z on ILC1, ILC2, ILC3/p, T cells, CD56^{bright} NK and CD56^{dim} NK cells. (B) Cumulative data representing both single and co-expression of CD28, GITR, ICOS or CD3z in different immune subsets.

FIG. 9. ST2 overexpression activates ILC2 and enhances their proliferation in response to IL-33 in vitro. (A) Schematic representation of the ST2 vector encoding the IL1RL1 gene and a ΔNGFR reporter gene. (B) Flow cytometry dot plot comparing transduced (NGFR⁺) and untransduced (NGFR⁻) ILC2s after transduction with ST2 vector. (C) Cumulative data showing the transduction efficiency with the ST2 vector (n=9). (D) Representative histogram and bar plot showing geometric mean fluorescence intensity (gMFI) of ST2 expression on NGFR⁺ (transduced) versus NGFR⁻ (untransduced) ILC2s (n=4). (E) Representative histograms showing surface expression of activation markers—CD69, CD86, ICOS, HLA-DR, and CD25—on ST2-overexpressing ILC2s and untransduced (UT) ILC2s after 6 hours of culture with IL-2 and IL-33. Bar plots demonstrate the fold change in expression of these markers relative to UT counterparts. (n=9). (F) Bar plots showing the fold change gMFI of intracellular cytokine production between ST2-overexpressing and UT-ILC2s after 6 hours of stimulation with IL-2, IL-7 and IL-33 (n=4-7). (G) Bar plots comparing secreted cytokine levels, measured by cytometric bead array, from ST2-overexpressing and UT-ILC2s after 6 hours of stimulation with IL-2, IL-7, and IL-33 (n=7 ). (H) Proliferation of transduced and UT-ILC2 cultured in expansion media (IL-2, IL-7, IL-33) over 7 days, measured by cell counts (n=7).

### DETAILED DESCRIPTION

In the following description, numerous specific details are set forth to provide a thorough understanding of the invention. However, it is understood that the invention may be practiced without these specific details.

In an aspect, there is provided an innate lymphoid cell (ILC) expressing a chimeric antigen receptor (CAR), the CAR comprising an antigen recognition domain for recognizing a target organ or target cell, a transmembrane domain, and a signalling domain for activating signalling within the ILC.

When expressed in a cell, the antigen recognition domain of the CAR is exposed to the outside of the cell and interacts with potential target molecules. Typically, the antigen-specific extracellular domain of a chimeric antigen receptor recognizes and specifically binds an antigen, for example, a surface-expressed antigen of a malignancy or a target tissue. An antigen-specific extracellular domain specifically binds an antigen when, for example, it binds the antigen with an affinity constant or affinity of interaction (KD) between about 0.1 pM to about 10 μM, preferably about 0.1 pM to about 1 μM, more preferably about 0.1 pM to about 100 nM. Methods for determining the affinity of interaction are known in the art. An antigen-specific extracellular domain suitable for use in a CAR of the present disclosure may be any antigen-binding polypeptide, a wide variety of which are known in the art. In some instances, the antigen-binding domain is a single chain Fv (scFv). Other antibody-based recognition domains (cAb VHH (camelid antibody variable domains) and humanized versions thereof, IgNAR VH (shark antibody variable domains) and humanized versions thereof, sdAb VH (single domain antibody variable domains) and “camelized” antibody variable domains are suitable for use. The design of antigen recognition domains to target specific cell types would be known to the skilled person in the art.

A chimeric antigen receptor of the present disclosure also comprises an intracellular signaling domain that provides an intracellular signal to the cell upon antigen binding to the antigen-specific extracellular domain. The intracellular signaling domain of a chimeric antigen receptor of the present disclosure is responsible for activation of at least one of the effector functions of the cell in which the chimeric receptor is expressed.

Typically, the antigen-specific extracellular domain is linked to the intracellular domain of the chimeric antigen receptor by a transmembrane domain. A transmembrane domain traverses the cell membrane, anchors the CAR to the cell surface, and connects the extracellular domain to the intracellular signaling domain, thus impacting expression of the CAR on the cell surface. Chimeric antigen receptors may also further comprise one or more costimulatory domain and/or one or more spacer/linker.

In an embodiment, the signalling domain comprises ST2(IL1RL1), CD28, CD122(IL2RB), CD132(IL2RG), CD127(IL7R), IL17RB(IL17RB), GITR (TNFRSF18), ICOS(ICOS), TNFR2(TNFRSF1B), 41BB(TNFRSF9), OX40(TNFRSF4) or CD3ζ(CD247) or a functional fragment thereof.

As used herein “fragment” relating to a polypeptide means a polypeptide consisting of only a part of the intact polypeptide sequence and structure, or the nucleotide sequence and structure, of the reference gene. The polypeptide fragment can include a C-terminal deletion and/or N-terminal deletion of the native polypeptide, or can be derived from an internal portion of the molecule. Similarly, a polynucleotide fragment can include a 3′ and/or a 5′ deletion of the native polynucleotide, or can be derived from an internal portion of the molecule. A functional fragment would retain at least partial activity of the intact polypeptide, for example, the ability to trigger signaling in the cell within which the CAR is expressed.

Preferably, the signalling domain comprises ST2 or a functional fragment thereof.

In some embodiments, the CAR further comprising a co-stimulatory domain. Preferably, the CAR co-stimulatory domain comprises CD28, GITR, ICOS, TNFR2, 41BB, OX40, or a functional fragment thereof.

### In Some Embodiments, the Target Organ or Target Cell Is Cancerous.

In some embodiments, the target organ or target cell is associated with transplantation of organs or cells. In these instances the target organ or target cell can be associated with hematopoietic stem cell transplantation. Alternatively, the target cell is donor derived T cells or recipient derived T cells.

In some embodiments, the ILC is group 2 innate lymphoid cell (ILC2). Preferably, the ILC2 is IL-10 producing.

In some embodiments, the ILC is a group 3 innate lymphoid cell (ILC3).

In some embodiments, the ILC is a group 1 innate lymphoid cell (ILC1).

In an aspect, there is provided the CAR-expressing ILC described herein for use in the treatment or prevention of, or in the manufacture of a medicament for use in the treatment or prevention of, cancer, an autoimmune disease, transplant rejection, Graft-versus-Host disease, an immunodeficiency disease or an inflammatory disease.

In an aspect, there is provided a method of treating or preventing in a subject, cancer, an autoimmune disease, graft-versus-host disease, an immunodeficiency disease or an inflammatory disease, the method comprising administering to the subject the CAR-expressing ILC described herein.

In an aspect, there is provided a chimeric antigen receptor as described herein.

In an aspect, there is provided a chimeric antigen receptor (CAR), the CAR comprising an antigen recognition domain for recognizing a target organ or target cell, a transmembrane domain, and an ST2 signalling domain.

In an aspect, there is provided a nucleic acid encoding the chimeric antigen receptor described herein.

In an aspect, there is provided a vector comprising the nucleic acid described herein. Preferably, the vector is a viral vector.

In an aspect, there is provided a cell expressing the any of the CAR described herein, wherein the cell is a T cell, regulatory T cell (Treg), a myeloid cell or a macrophage, a dendritic cell (DC), a Natural Killer (NK) cell or a B cell. In a further aspect, there is provided a pharmaceutical composition comprising the CAR expressing cells described herein along with a pharmaceutically acceptable carrier.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

The advantages of the present invention are further illustrated by the following examples. The examples and their particular details set forth herein are presented for illustration only and should not be construed as a limitation on the claims of the present invention.

### EXAMPLES

### Methods and Materials

### Cite-Seq Staining of PBMCs

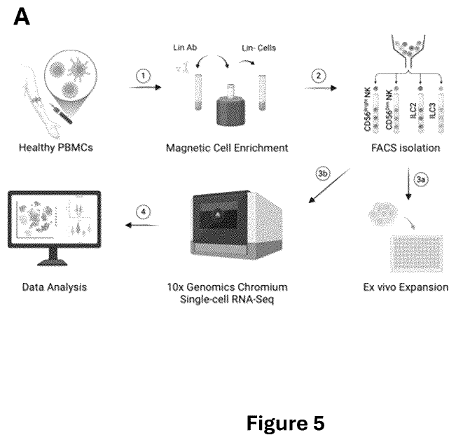

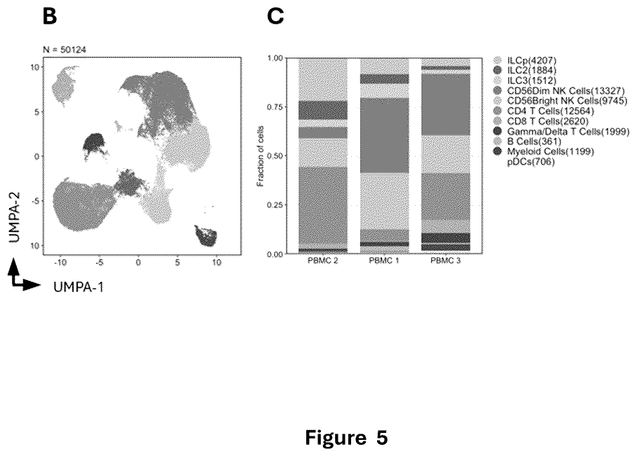

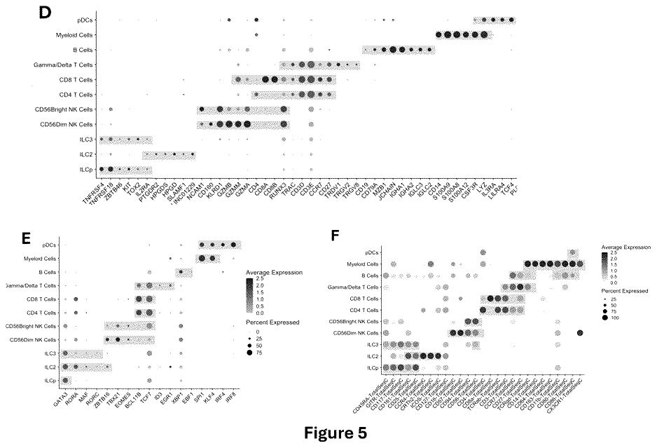

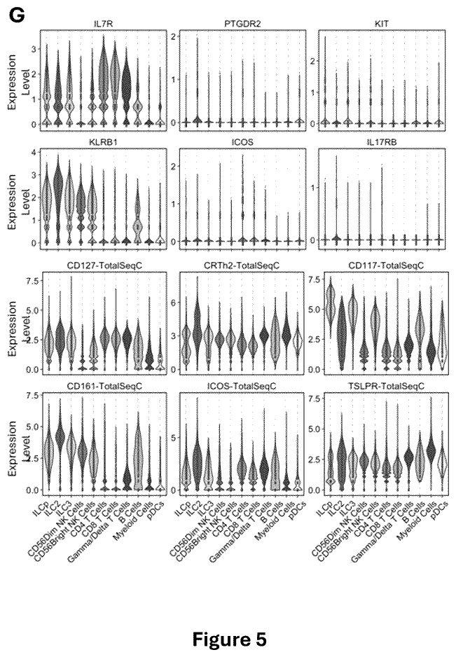

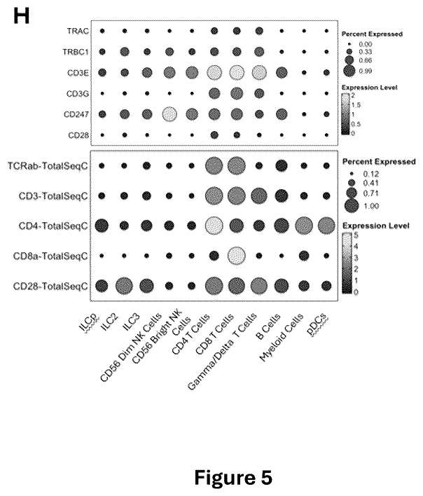

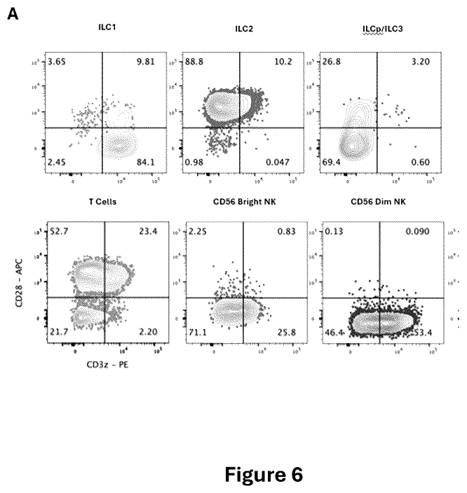

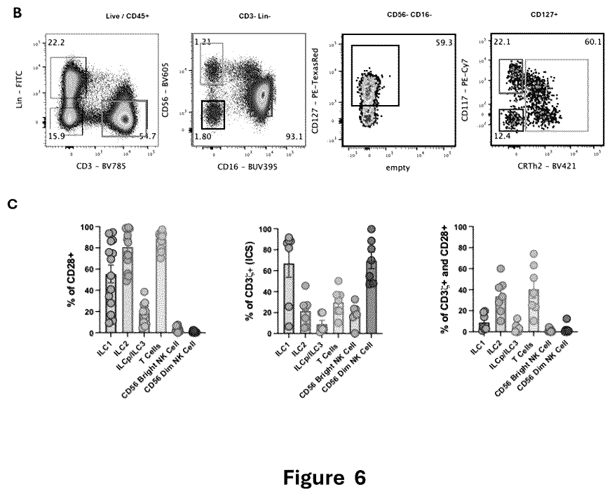

Due to the low abundance of ILCs in peripheral blood mononuclear cells (PBMCs) and the substantial overlap between ILCs and T cells in both protein and transcriptomic profiles, we performed targeted enrichment of both cell types. To block non-specific antibody binding, TruStain FcX™ Fc Blocking reagent (5 μL) was added to each sample, followed by a 10-minute incubation at 4 °C. T cells were enriched using the EasySep™ Human T Cell Isolation Kit (StemCell Technologies), and CITE-seq was subsequently performed on these samples in parallel. For ILC enrichment, a lineage cocktail containing FITC-conjugated antibodies targeting B cells, T cells, and myeloid cells was used to deplete lineage-positive cells. Antibodies were used for lineage exclusion. Cells were washed, resuspended in EasySep Buffer, and enriched using the EasySep™ FITC Positive Selection Kit II (StemCell Technologies) according to the manufacturer's instructions. Following enrichment, cells were stained with flow cytometry antibodies along with the TotalSeq™-C and unique hashtag antibody cocktail (1:1 ratio), and sorted. All TotalSeq™-C antibodies were used. Cells were sorted into CD56\hat{ }dim NK cells, CD56\hat{ }bright NK cells, or helper ILCs. For PBMC samples, sorted NK cells were pooled into one tube containing equal proportions of CD56\hat{ }dim and CD56\hat{ }bright NK cells, while helper ILCs were collected in a separate tube. Each population was washed three times, resuspended at 1,200 cells/μL in PBS with 0.05% BSA, and submitted for sequencing. Referring to FIGS. 5 and 6, there is shown Human peripheral blood ILC2 highly express CD28 and CD3ζ.

### Cite-Seq Data Analysis Pipeline

Single-cell RNA sequencing was performed using the 10× Genomics Single Cell 5′ v2 platform following the manufacturer's instructions, with a target capture of 12,000 cells per sample. Reverse transcription, cDNA amplification, and library preparation were carried out using the 10× Genomics Single Cell 5′ v2 reagents. Libraries were sequenced to a target depth of 40,000 reads per cell. Raw sequencing data were processed using the Cell Ranger pipeline (version 6.1.2) for read alignment to the GRCh38/hg38 human reference genome and generation of gene expression matrices. Data analysis was conducted in R using the Seurat package. Individual Seurat objects were created for each sample. Quality control for splenic samples included filtering out cells with >10% mitochondrial UMIs, fewer than 500 or more than 5,000 detected genes, and fewer than 500 or more than 20,000 total UMIs. For PBMC samples, cells with >5% mitochondrial UMIs, <500 or >5,000 detected genes, and <500 or >30,000 total UMIs were excluded to remove low-quality cells and potential doublets. SCTransform was used for normalization of individual PBMC datasets, which were subsequently integrated using the Harmony algorithm (RunHarmony). Dimensionality reduction was performed with principal component analysis (RunPCA), and clustering was achieved using the Louvain algorithm based on the top 50 principal components (FindNeighbors, FindClusters). Visualization was done using UMAP. Cell type annotation was based on the top differentially expressed genes and/or surface proteins identified using FindMarkers and confirmed through manual curation. Gene set enrichment analysis (GSEA) was conducted using ranked gene lists generated by the Wilcoxon rank-sum test, with reference to curated gene sets.

### Flow Cytometry and FACS Sorting

Following in vivo experiments, spleen, lungs and liver tissues were chopped with a scalpel blade and digested in FACS buffer containing 25 μg/ml DNase I and 2% FBS and passed through a cell strainer. Red blood cells in blood samples and tissues were lysed with ACK buffer. Cells from in vitro experiments were washed with FACS buffer (PBS+2% FCS). Washed cells were incubated 15 min with human TruStain FcX (BioLegend) and then stained for cell surface markers and Fixable viability dyes AF700 (ThermoFisher, France) were used and incubated for 30 min at 4 degrees. For intracellular staining, cells were fixed and permeabilized using FOXP3/Transcription Factor Staining set (eBioscience) and incubated with intracellular antibodies at 4 degrees for 30 min. White blood cells were harvested from healthy HLA-A*02-negative Human Peripheral Blood Leukopak and aliquoted. Thawed aliquotes washed with FACS buffer containing 25 μg/ml DNase and stained with human TruStain FcX (BioLegend) and incubated with a cocktail of lineage antibodies conjugated to the FITC. Cells are washed in FACS buffer, resuspended in EasySep Buffer (StemCell Technologies) and enriched using the EasySep FITC Positive Selection Kit II (StemCell Technologies) per manufacturer instructions. Enriched cells were stained with antibody cocktail as described above. and sorted using either FACSAria Fusion or Symphony A5(BD Biosciences).

### Production of Lentivirus for the Establishment of CAR-ILC2s

The HEK293T cells were cultured in Iscove Modified Dulbecco Media (IMDM) base medium supplemented with 10% fetal bovine serum (FBS), 1% penicillin streptomycin, and 1% Glutamax. One day prior to transfection, the HEK 293T cells were replated in 15 cm cell culture plates with fresh media and were left to incubate at 37 °C overnight. The next day, the confluency of the cells was visualized by a microscope, and the cells were transfected only if they were 70-80% confluent, as the confluency of the cells affects transfection efficiency. The calcium-phosphate precipitated method of transfection was used to prepare the DNA transfection media. Appropriate amounts of transfer, packaging (PKG), envelope (ENV) and regulatory (REV) vectors were combined into a tube containing HEPES buffered solution and 0.5 M calcium chloride. This DNA mixture was then transferred dropwise to another tube containing 2× HEBS solution (280 mM NaCl, 50 mM HEPES, 1.5 mM Na2HPO4). The transfection mixture was left to incubate at room temperature for 20 minutes and was then added to the HEK 293T cells dropwise. The cells were incubated at 37 °C for 12-16 hours. The next day, the supernatant was collected, and fresh complete IMDM growth medium containing 1 mM sodium butyrate (NaBut) was added. The cells were left to incubate overnight, prior to harvest and concentration of produced lentivirus. Lentivirus titration. and Flowcytometry plots representing lentivirus titration using HEK293T cells. Different dilution of concentrated virus were added to HEK293T cells. Viral titrations were calculated after 72 hours incubation at 37 °C using flowcytometry. Optimization of lentiviral transduction for human ILC2 was tested at varying MOIs: 0, 1, 2, 5, 7, 10, 15 and 20 and different time point. Transduction efficiency was determined based on percentage of NGFR positive live ILC2s.

### Generation of Human ILC2 with HLA-A*02-Specific CAR Constructs

Sorted human ILC2s were cultured in complete X-VIVO 15 media (Lonza) supplemented with 5% human AB serum (Sigma), 100U/mL Penicillin-Streptomycin (Gibco) and 1× GlutaMAX (Gibco). ILC2s were expanded using 100 IU/mL IL-2 (SteriMax), 20 ng/mL IL-7 and 20 ng/mL IL-33 (BioLegend). Isolated ILC2s were expanded for two consecutive weeks to keep the cells in an exponential phase of growth and cells were replenished every other day with 100 IU/mL IL-2, and 20 ng/mL IL-7. After two weeks, an aliquate of ILC2 was stimulated with Cell Stimulation Cocktail(eBioscience) and screened for IL-10 production. IL-10 producing ILC2s were selected and transduced with lentiviral particles at a MOI of 5-7. Transduced ILC2s were incubated for 7 days, and cells were replenished every other day with IL-2 and IL-7. Transduced cells were sorted seven days later.

### Supernatant Cytokine Assessment

For secreted factor analysis, plasma or supernatants were collected from various condition and stored at −80 °C. Analytes were measured using the 12-plex LegendPlex Human Th Cytokine Panel(BioLegend) or the 13-plex LegendPlex Human Proinflammatory Chemokine Panel 1(BioLegend) on thawed supernatant samples per manufacturer's instructions.

### In Vitro Assays

For in vitro experiments, each ILC2 donor constituted an individual experiment, and n represents the number of ILC2 donors. Per experiment, 3 to 4 different ILC2 were used (biological replicates). For baseline assessment of CAR-ILC2s, CAR-ILC2s and UT-ILC2s were expanded in ILC2 expansion media for 24 or 72 hours and assessed for bassline surface marker, transcription factors and cytokine secretion. For activation and functional assays, CAR-ILC2s were co-cultured with the HLA-A2+K562 cells or HLA-A2+ T cells for 24 hours. ILC2s were stimulated for 24 h with IL-33 as a positive control while control negative UT-ILC2s were incubated in IL-2 for 24 hours. For T cell suppression assay, allogeneic CD4 and CD8 naïve T cells were stimulated with anti-CD3/CD28-coated dynabeads (#40203D, Life Technologies, 1 bead:1 cell ratio) and then co-cultured with CAR for 24 hours. For control positive condition, stimulated T cells incubated in IL-2 alone and for negative condition stimulated T cells co-cultured with UT-ILC2s.

### Xenogeneic GvHD Mouse Model

Six-to-8-week-old NOD.Cg-PrkdcscidIl2rgtm1Wjl/SzJ(NSG) mice were used with a median (range) weight of 22 g (20-25 g). Mice were housed in a specific opportunistic pathogen-free facility, in individually, positively ventilated polysulfone cages with HEPA-filtered air, controlled 12 h light/dark cycle, temperature of 20-26 °C, and relative humidity of 30-70%. Filtered tap water and standard rodent chow were provided ad libitum. For experiments, mice were age-matched but distributed randomly to treatment groups. Mice given 250 cGy irradiation on one day prior to intravenous tail vein injection of 5×106 PBMCs with or without 5×106 expanded allogeneic CAR-ILC2 and UT-ILC2. Control mice were injected with PBS. Mice were monitored daily for symptoms of GVHD including weight loss, fur loss and skin inflammation, hunch, activity, and pain, scored on a scale of 0-3. At endpoint, blood, bone marrow, spleens, lungs and liver were harvested as previously described

### RESULTS

### Engineering ILC2s Using Chimeric Antigen Receptor

We previously developed a novel method to isolate and expand human IL-10-producing ILC2s from healthy human PBMCs (see WO2023/245283 and WO2024/243679). This method offers advantages in terms of efficiency and yield. Further, we have demonstrated in two humanized animal models that cell therapy with human IL-10-producing ILC2s protect mice against xenoGVHD and allo-islet transplant rejection¹⁹ (). Importantly, our work provides a framework for expanding ILC2s in a reproducible manner, ensuring their functionality and stability for therapeutic applications. This proprietary method for generating and expanding stable regulatory ILC2s represents a significant opportunity for developing cell-based therapies tailored to individual patients.

### CAR-ILC2s: A Novel Approach to Enhancing Regulatory Cell Therapy

Recognizing the benefits of IL-10-producing ILC2s, we undertook genetic engineering to overcome limitations related to long-term persistence and target tissue homing in ILC2s. The concept of developing chimeric antigen receptor ILC2s (CAR-ILC2s) to enhance specificity, durability, and efficacy is novel and to our knowledge has not been reported. CAR-ILC2s would be engineered to express a chimeric receptor that targets specific antigens allowing them to exert their immunosuppressive effects in a more controlled and localized manner. Coupled with non-antigen dependent activation of ILC2 in inflamed tissue, CAR constructs guide ILC2 to target specific targets to exert their effect directly on the source of reactions such as allogeneic T cells in alloHSCT and transplanted organ.

To assess the potency of CAR-ILC2s, we utilized the allo-antigen system used in proof of principal studies with Tregs. This HLA-A2-targeting CAR system was successfully previously applied in CAR-Tregs and a humanized version of this CAR system being used in clinical trials. The HLA-A2 CAR design concept has been adapted for our ILC2 platform.

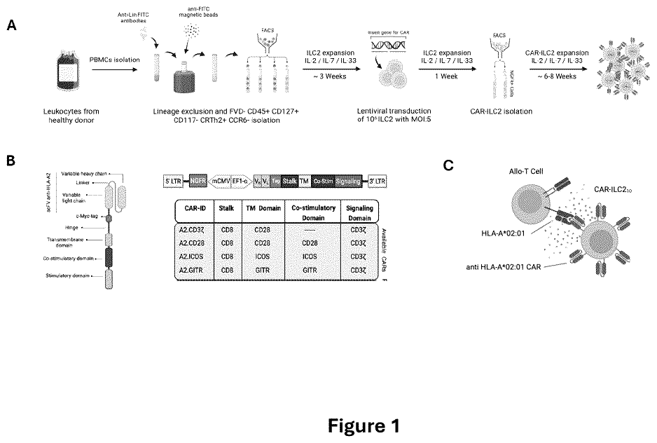

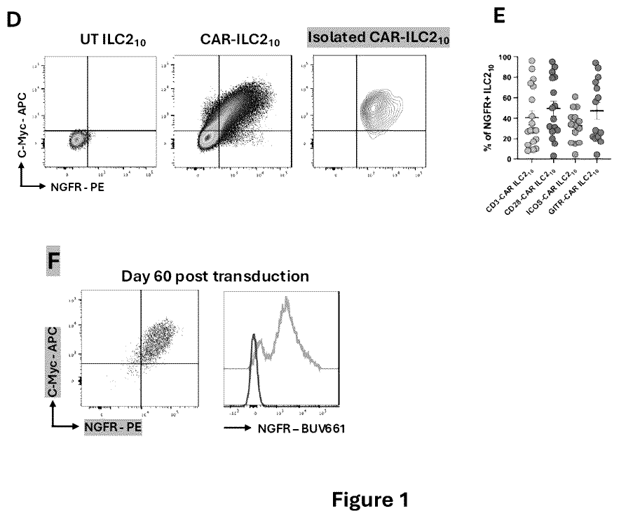

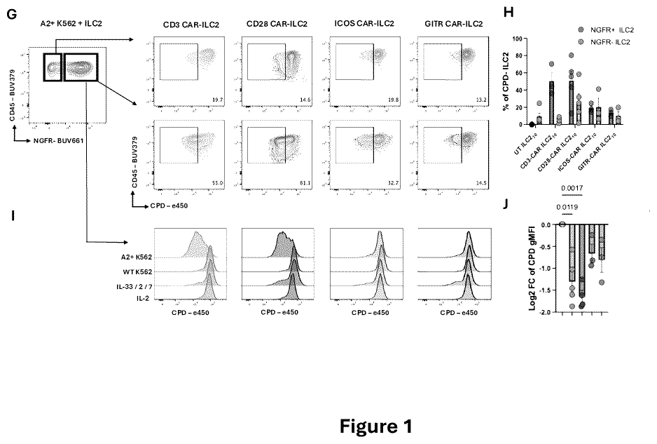

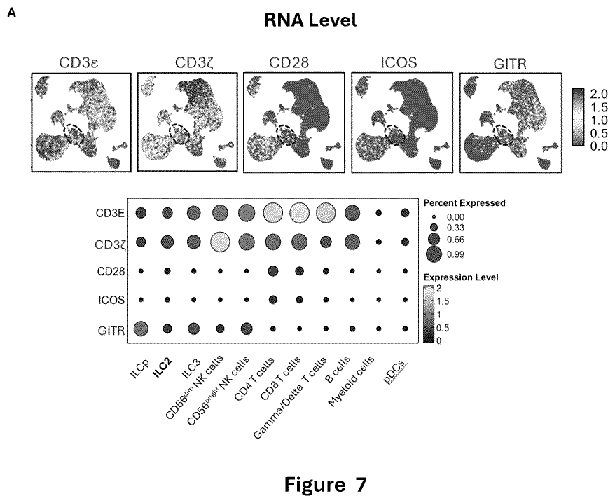

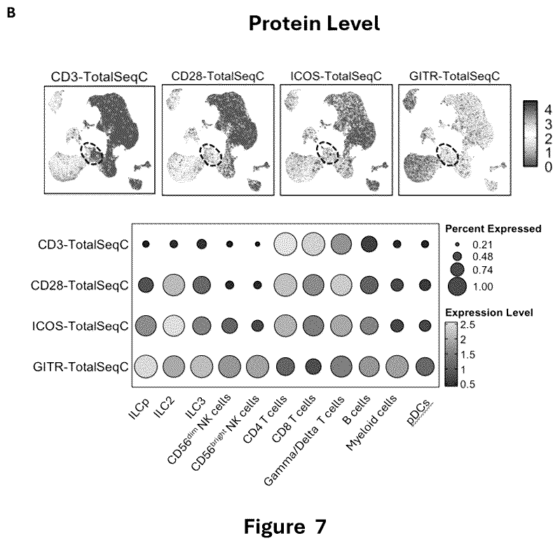

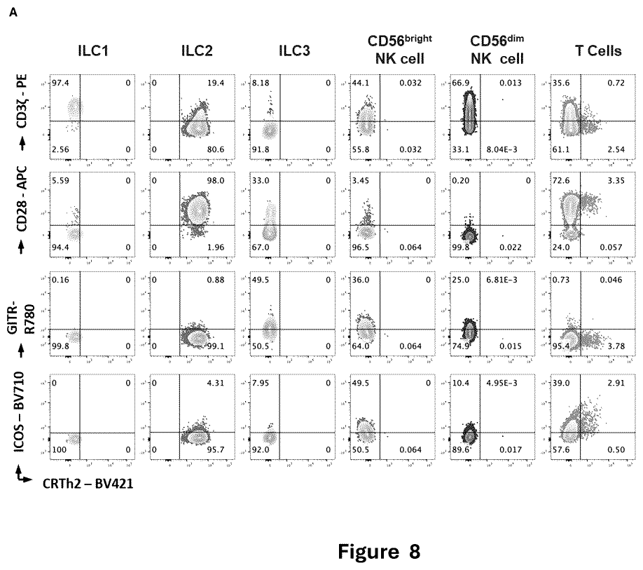

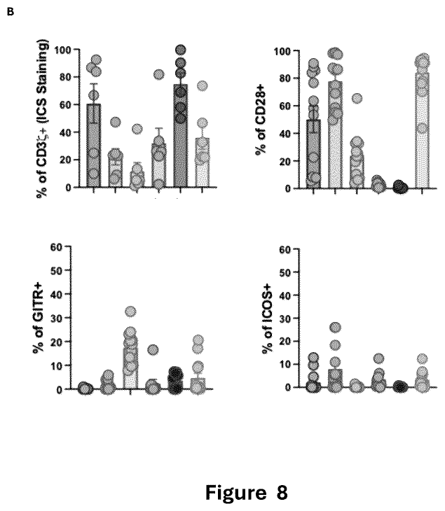

Based on screening ex vivo and expanded human ILC2 using CITEseq, we observed that ILC2s express ICOS, GITR, CD28, and CD3ζ (FIG. 1). Referring to FIG. 7, there is shown human peripheral blood ILC2 express GITR and ICOS, in addition to CD28 and CD3z. Referring to FIG. 8, there is also shown a representative flow cytometry dot plot demonstrating co-expression of CRTH2 and CD28, GITR, ICOS or CD3z on ILC1, ILC2, ILC3/p, T cells, CD56^{bright} NK and CD56^{dim} NK cells. FIG. 6 also shows cumulative data representing both single and co-expression of CD28, GITR, ICOS or CD3z in different immune subsets. Therefore, we selected ICOS, GITR, CD28, and CD3ζ as co-stimulatory domains for our CAR constructs in ILC2s. Although the role of ICOS and GITR in ILC2 function has been studied, there are no reports on CD28 and CD3ζ in ILC2s. Each of these molecules could impact ILC function and stability:
ICOS (Inducible T-cell Costimulator): Supports survival, proliferation, and homeostasis of ILC2s^{9,20-23}.
GITR (Glucocorticoid-Induced TNFR-Related Protein): Enhances suppressive activity and persistence in inflammatory environments^{24,25}.
CD28: Essential for activation, expansion, and sustained function in T cells²⁶.
CD3ζ (signalling domain in first-generation CAR): Provides robust activation signals for functionality of a variety of cells²⁷.

The gene encoding the desired CAR (e.g., anti-HLA-A2 CAR with specific co-stimulatory domains like CD28 and CD3ζ) were designed and cloned into a third-generation lentiviral transfer plasmid as reported in prior publications and obtained. Using this lentiviral system, we transduced human ILC2s. Here, ILC2s were isolated from PBMCs using our established flow cytometry sorting protocol and expanded for two weeks with IL-2, IL-7, and IL-33, then were then transduced with the lentivirus, resulting in stable integration of the CAR gene. The transduced CAR-ILC2s were further expanded in vitro with cytokines indicated above to obtain sufficient cells for downstream applications, and CAR expression on their surface was confirmed by flow cytometry (FIG. 1)

### Validation and Therapeutic Potential of CAR-ILC2s

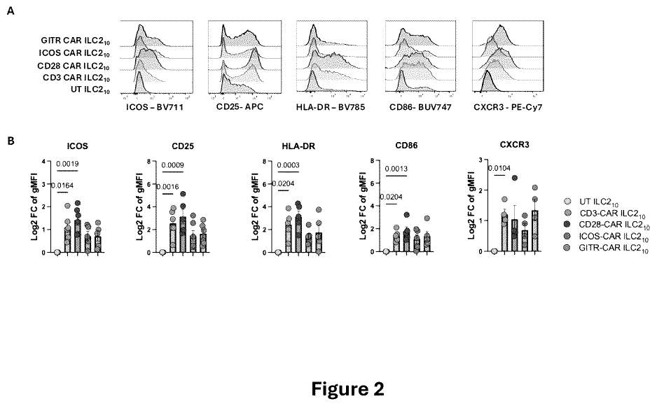

We have successfully generated functional human ILC2s expressing CAR constructs, demonstrating that CAR expression is stable over an extended period (FIG. 2). The engineered CAR-ILC2s maintained their identity and functionality, confirming the feasibility of utilizing CAR signaling to enhance ILC2-based regulatory cell therapy.

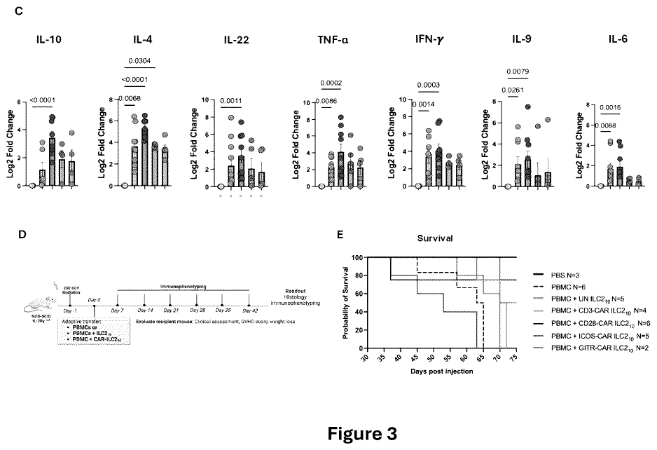

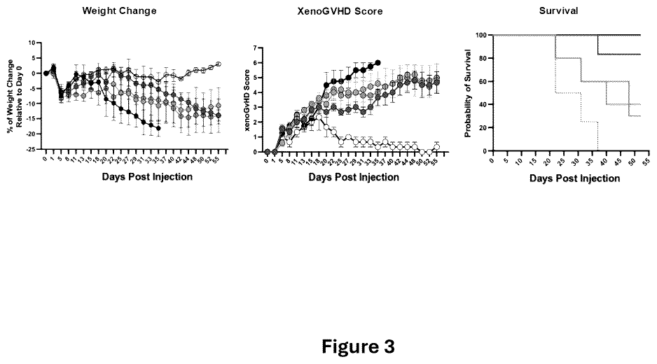

To assess functionality of CAR system within human ILC2s, we conducted co-culture experiments with target cells expressing HLA-A2 antigens. CAR signaling led to robust activation of ILC2s, enhancing their proliferation and cytokine production (FIG. 2). Specifically, CAR engagement triggered the expansion of ILC2s and promoted the secretion of key ILC2 cytokines IL-4, IL13, as well as IL-10 from IL-10-producing ILC2s (ILC2₁₀) (FIG. 3). Similar activation was also observed with IL-10-ILC2s. These results confirmed that CAR signaling can support the immunoregulatory properties of ILC2₁₀, allowing them to exert targeted effects on immune responses. In in vivo studies, we tested the therapeutic potential of CAR-ILC2₁₀ in a xenogeneic graft-versus-host disease (xenoGVHD) model commonly used to assess therapies for GVHD and transplantation. CAR-ILC2₁₀ incorporating both CD28 and CD3ζ co-stimulatory domains demonstrated significantly improved survival in treated mice (FIG. 3), indicating their enhanced persistence and/or function in an inflammatory environment. CD28 signaling played a critical role in sustaining ILC2₁₀-mediated immune modulation, allowing for prolonged regulatory effects (FIGS. 2,3)

### Discussion

To our knowledge, there are no previous reports on engineered human ILC2s, and we are the first to demonstrate the feasibility of CAR-ILC2₁₀ therapy. Our findings provide surprising and compelling evidence that CAR-ILC2s can be harnessed to mitigate immune-driven pathologies, emphasizing the importance of optimizing co-stimulatory domains for durable therapeutic efficacy.

By leveraging CD28, ICOS, GITR, and CD3ζ in our novel CAR-ILC2 system, we have developed a platform to investigate their distinct roles in immune regulation and fine-tune their therapeutic potential. While CD28, ICOS, GITR, and CD3ζ are classical T cell co-stimulatory and signaling molecules, they are also variably expressed and functional in ILC2s and there was no suggestion whether they would induce IL-10 production. The specific combinations of these co-stimulatory domains result in enhanced function of the CAR-ILC2s. The ability to generate stable, functional CAR-ILC2s opens new possibilities for treating autoimmune diseases, promoting transplantation tolerance, inhibiting harmful inflammation and facilitating tissue repair. Patient-derived CAR-ILC2s could also be used as an off-the-shelf or personalized therapy to suppress inflammation while maintaining tissue integrity. Our findings establish a new frontier in regulatory cell therapy, demonstrating that engineered ILC2s can serve as a powerful tool for immune modulation. These advancements may lead to a novel class of cell-based immunotherapies with broad applications in immune tolerance, transplantation, and regenerative medicine.

### Applications for Cancer Immunotherapies

The described CAR-ILC2s and specifically CARs in IL-10-producing ILC2s (ILC2₁₀), could also be used to harness conventional or inflammatory ILC2s, which are currently being explored for applications in cancer immunotherapies. CAR-ILC2s could be used to activate inflammatory functions in these inflammatory ILC2 populations.

### CAR-ILC2s with ST2 Signaling Domain

ST2, also known as IL1RL1, is a key component of the IL-33 signaling axis and is highly expressed by regulatory T cells (Tregs) and ILC2s²⁸. In Tregs, ST2 plays a crucial role in maintaining immune homeostasis by enhancing their suppressive function and promoting tissue repair²⁹⁻³¹. IL-33 stimulation through ST2 has been shown to expand Tregs and reinforce their ability to mitigate excessive immune responses, making it a valuable target for immunomodulatory therapies. Similarly, ILC2s rely on ST2 signaling for their activation and function, particularly in tissue repair and the regulation of inflammatory responses³². These cells are essential for maintaining mucosal and epithelial integrity, as well as modulating immune reactions in response to damage or infection. The concept of integrating ST2 signaling into CAR constructs to exploit these immunoregulatory properties for non-cytotoxic applications is novel and represents a potential area for intellectual property development. IL-33 has been shown to enhance IL-10 production by ILC2s, promoting their anti-inflammatory function. Therefore, IL-10-producing ILC2s have been identified as key regulators of immune responses, capable of suppressing excessive inflammation and facilitating tissue repair. We have recently reported that IL-10-producing ILC2s suppressed the development of humanized mouse models of graft-versus-host disease and islet transplant, supporting applications in cell therapy for transplant, autoimmunity and inflammation³³.

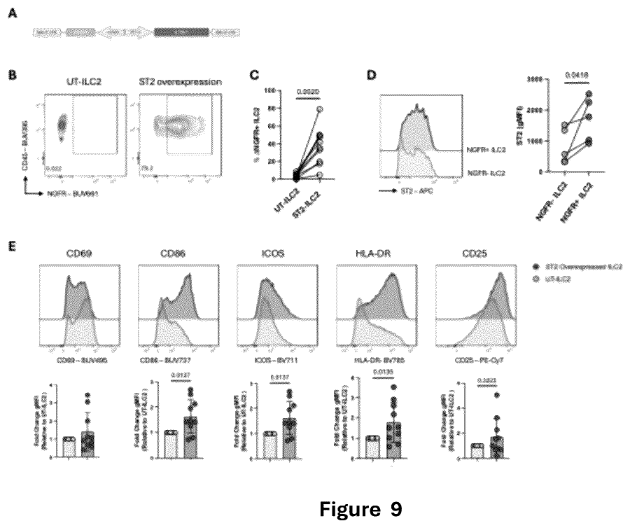

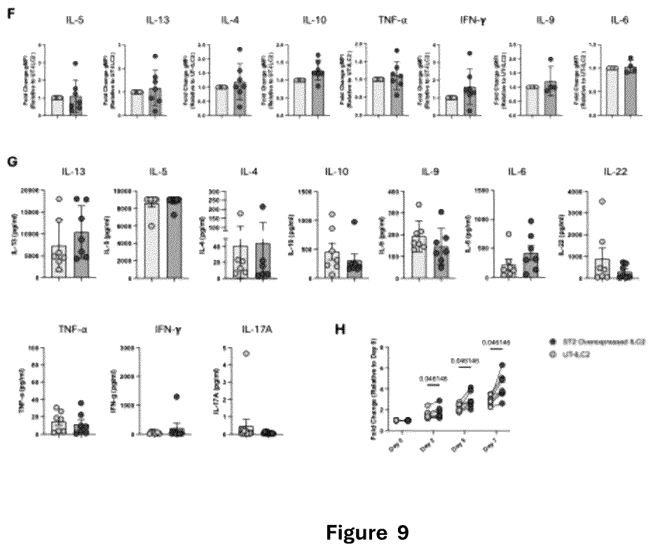

Referring to FIG. 9, we show that ST2 overexpression activates ILC2 and enhances their proliferation in response to IL-33 in vitro.

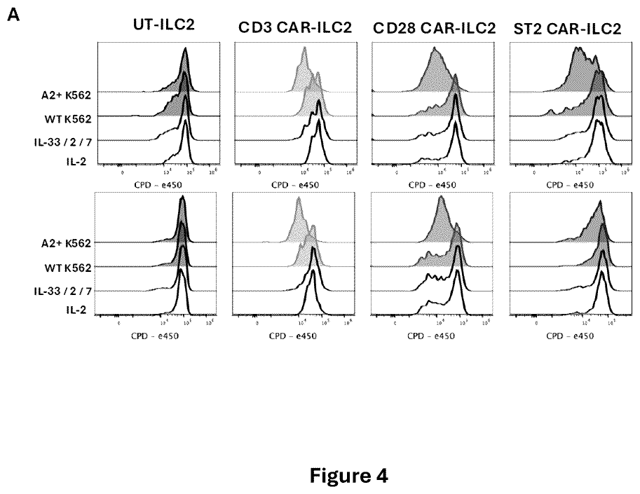

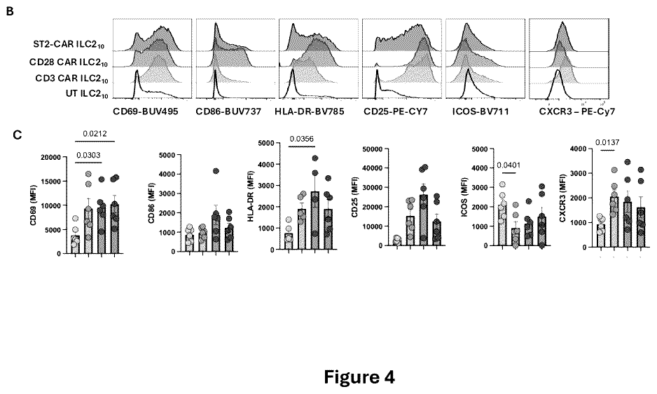

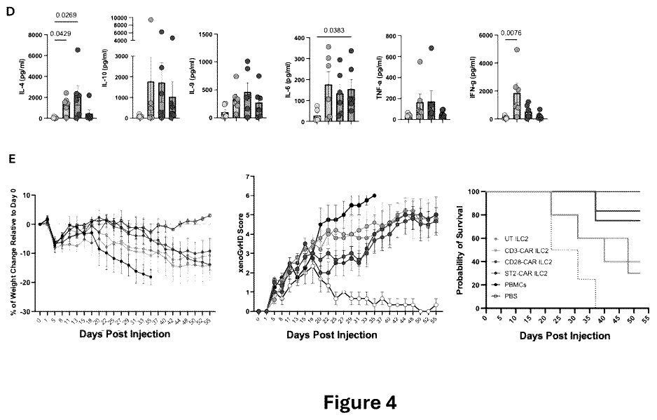

Following our above development of CAR-ILC2s and our identification of co-stim domains that could activate human ILC2s, we have now designed a novel ST2-CAR, whereby we insert and ST2 signaling domain to trigger ILC2 activation following CAR recognition of antigen. The ST2-CAR was designed in silico based on the anti-HLA-2 CAR plasmids described above, with modifications specific to incorporating the ST2 signaling domain. The parent CARs were linearized, and unnecessary domains were removed using inverse PCR (iPCR). A designed ST2 insert with homologous ends was synthesized and introduced into the parent anti-HLA-A2 CARs using seamless integration. Successfully recombined colonies expressing ST2-CAR were selected using colony screening PCR, and their sequences were validated through whole plasmid sequencing. The verified ST2-CAR plasmid was propagated via maxiprep for downstream applications. We have successfully produced lentiviruses encoding our novel ST2-CAR using a lentiviral system we have used in ILC2s and used in Tregs, and demonstrated successful transduction of human ILC2s with this construct (FIG. 4). Expression and functionality of ST2-CAR were then measured in vitro and in vivo.

We describe herein a first-generation anti-HLA-A2 CAR engineered with an ST2 signaling domain, designed for use in cell-based immunotherapy. This specific CAR construct, targeting HLA-A2 and incorporating the ST2 signaling domain, is a novel entity. This CAR construct can be introduced into ILC2s and Tregs to leverage their immunomodulatory properties, distinguishing it from conventional cytotoxic CAR-T therapies. This CAR product prioritizes immune modulation over direct cytotoxicity, potentially expanding its therapeutic applications beyond oncology. The ST2 signaling pathway is central to immune regulation, tissue repair, and homeostasis, providing a unique mechanism to control inflammation and promote immune tolerance. We describe herein a cell-based therapy using ST2-CARs that enhances tissue homeostasis and prevents inflammation, offering new strategies for managing immune-mediated disorders, transplantation, and autoimmune diseases. ST2-CAR represents a novel approach to CAR therapy by adding a specific signalling domain that activates ILC2s. This CAR construct would also have potential applications in Tregs, where ST2-expressing Tregs can promote tissue repair. Unlike traditional CAR constructs that utilize signaling domains from CD3ζ, CD28, 4-1BB (CD137), or OX40(CD134 ) to enhance immune cell expansion and cytotoxicity, our ST2-CAR harnesses the unique IL-33/ST2 axis in a CAR context, which is a key differentiator.

ST2-CAR ILC2s and ST2-CAR Tregs are two cell therapy products based on our novel ST2-CAR construct. Engineering ILC2s with an ST2-CAR could augment their regenerative and anti-inflammatory properties, offering a promising therapeutic approach for inflammatory diseases, tissue damage such as that which occurs with ischemia reperfusion injury, fibrotic disorders, and autoimmune conditions. These engineered cells could be directed to inflamed tissues, where they would not only suppress excessive immune activation but also promote tissue repair and regeneration. ST2 expression in Tregs is linked to their stability, suppressive function, and survival in inflammatory environments. Introducing ST2-CAR into Tregs could enhance their ability to home to inflamed tissues and exert localized immune suppression and tissue-reparative functions. These ST2-CAR Tregs could be utilized in transplantation to prevent graft rejection, in autoimmune diseases to suppress autoreactive immune responses, and in inflammatory disorders to mitigate excessive immune activation. By leveraging the IL-33/ST2 axis, ST2-CAR Tregs could achieve long-term immune regulation without the need for continuous immunosuppressive therapy, reducing the risk of systemic side effects. The ability to engineer ILC2s and Tregs with ST2-CAR introduces a new class of cell-based therapies that in addition to immune modulation, could promote tissue protection/repair, and inflammatory resolution, representing a significant advancement in cellular immunotherapy.

Our non-humanized ST2-CAR is also a valuable tool for preclinical research and non-human model systems. The insights gained from preclinical studies using this novel ST2-CAR construct would be instrumental in developing a humanized version optimized for clinical use, potentially leading to new CAR-based therapies for autoimmune diseases, transplantation tolerance, and tissue repair.

### Additional Applications of ST2-CARs in Anti-Cancer Immunotherapies

Further, ST2-CARs could be used to promote anti-cancer properties of inflammatory ILC2s, which are currently being examined for applications in anti-cancer therapy. Therefore, while we have shown they enhance ILC2₁₀ function, they would also enhance activation of ILC2s that could promote anti-tumor immunity or directly participate in anti-tumor killing.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### Reference List

1. Mamo, T. et al. Regulatory T Cells: A Review of Manufacturing and Clinical Utility. Transfusion (Paris) 62, 904 (2022).
2. Raffin, C., Vo, L. T. & Bluestone, J. A. Treg cell-based therapies: challenges and perspectives. Nature Reviews Immunology 2019 20:3 20, 158-172 (2019).
3. Arjomandnejad, M., Kopec, A. L. & Keeler, A. M. CAR-T Regulatory (CAR-Treg) Cells: Engineering and Applications. Biomedicines 10, (2022).
4. MacDonald, K. G. et al. Alloantigen-specific regulatory T cells generated with a chimeric antigen receptor. 126, (2016).
5. Sicard, A. et al. Donor-specific chimeric antigen receptor Tregs limit rejection in naive but not sensitized allograft recipients. American Journal of Transplantation 20, 1562-1573 (2020).
6. Wardell, C. M. et al. Short Report: CAR Tregs mediate linked suppression and infectious tolerance in islet transplantation. bioRxiv 2024.04.06.588414 (2024) doi:10.1101/2024.04.06.588414.
7. Dawson, N. A. J. et al. Systematic testing and specificity mapping of alloantigen-specific chimeric antigen receptors in regulatory T cells. JCI Insight 4, (2019).
8. Ellis, C. E. et al. Human A2-CAR T Cells Reject HLA-A2+Human Islets Transplanted Into Mice Without Inducing Graft-versus-host Disease. Transplantation 107, E222-E233 (2023).
9. Proics, E. et al. Preclinical assessment of antigen-specific chimeric antigen receptor regulatory T cells for use in solid organ transplantation. Gene Therapy 2022 30:3 30, 309-322 (2022).
10. Search for: CAR Treg|Card Results|ClinicalTrials. gov. https://clinicaltrials.gov/search?intr=CAR%20Treg.
11. Murphy, J. M., Ngai, L., Mortha, A. & Crome, S. Q. Tissue-Dependent Adaptations and Functions of Innate Lymphoid Cells. Frontiers in Immunology vol. 13 Preprint at https://www.frontiersin.org/articles/10.3389/fimmu.2022.836999 (2022).
12. Seehus, C. R. et al. Alternative activation generates IL-10 producing type 2 innate lymphoid cells. Nat Commun 8, (2017).
13. Bando, J. K. et al. ILC2s are the predominant source of intestinal ILC-derived IL-10. J Exp Med 217, (2020).
14. Kim, H. S. et al. A novel IL-10-producing innate lymphoid cells (ILC10) in a contact hypersensitivity mouse model. BMB Rep 49, 293 (2016).
15. Neumann, C. et al. Role of Blimp-1 in programing Th effector cells into IL-10 producers. J Exp Med 211, 1807-1819 (2014).
16. Reid, K. T. et al. Cell therapy with IL-10-producing group 2 innate lymphoid cells suppresses Graft-versus-Host disease. bioRxiv 2023.08.21.554158 (2023) doi:10.1101/2023.08.21.554158.
17. Golebski, K. et al. Induction of IL-10-producing type 2 innate lymphoid cells by allergen immunotherapy is associated with clinical response. Immunity 54, 291-307.e7 (2021).
18. Howard, E. et al. IL-10 production by ILC2s requires Blimp-1 and cMaf, modulates cellular metabolism, and ameliorates airway hyperreactivity. J Allergy Clin Immunol 147, 1281-1295.e5 (2021).
19. Reid, K. T. et al. Cell therapy with human IL-10-producing ILC2s limits xenogeneic graft-versus-host disease by inhibiting pathogenic T cell responses. Cell Rep 44, 115102 (2025).
20. Maazi, H. et al. ICOS:ICOS-Ligand Interaction Is Required for Type 2 Innate Lymphoid Cell Function, Homeostasis, and Induction of Airway Hyperreactivity. Immunity 42, 538-551 (2015).
21. Kamachi, F., Isshiki, T., Harada, N., Akiba, H. & Miyake, S. ICOS promotes group 2 innate lymphoid cell activation in lungs. Biochem Biophys Res Commun 463, 739-745 (2015).
22. Hrusch, C. L. et al. ICOS protects against mortality from acute lung injury through activation of IL-5+ ILC2s. Mucosal Immunol 11, 61-70 (2018).
23. Paclik, D., Stehle, C., Lahmann, A., Hutloff, A. & Romagnani, C. ICOS regulates the pool of group 2 innate lymphoid cells under homeostatic and inflammatory conditions in mice. Eur J Immunol 45, 2766-2772 (2015).
24. Galle-Treger, L. et al. Costimulation of type-2 innate lymphoid cells by GITR promotes effector function and ameliorates type 2 diabetes. Nat Commun 10, 713 (2019).
25. Nagashima, H. et al. GITR cosignal in ILC2s controls allergic lung inflammation. Journal of Allergy and Clinical Immunology 141, 1939-1943.e8 (2018).
26. Esensten, J. H., Helou, Y. A., Chopra, G., Weiss, A. & Bluestone, J. A. CD28 costimulation: from mechanism to therapy. Immunity 44, 973 (2016).
27. Bridgeman, J. S. et al. CD3ζ-based chimeric antigen receptors mediate T cell activation via cis- and trans-signalling mechanisms: implications for optimization of receptor structure for adoptive cell therapy. Clin Exp Immunol 175, 258 (2014).
28. Kakkar, R. & Lee, R. T. The IL-33/ST2 pathway: therapeutic target and novel biomarker. Nature Reviews Drug Discovery 2008 7:10 7, 827-840 (2008).
29. Spath, S., Roan, F., Presnell, S. R., Höllbacher, B. & Ziegler, S. F. Profiling of Tregs across tissues reveals plasticity in ST2 expression and hierarchies in tissue-specific phenotypes. iScience 25, 104998 (2022).
30. Pastille, E. et al. The IL-33/ST2 pathway shapes the regulatory T cell phenotype to promote intestinal cancer. Mucosal Immunology 2019 12:4 12, 990-1003 (2019).
31. Hemmers, S., Schizas, M. & Rudensky, A. Y. T reg cell-intrinsic requirements for ST2 signaling in health and neuroinflammation. J Exp Med 218, e20201234 (2020).
32. Topczewska, P. M. et al. ILC2 require cell-intrinsic ST2 signals to promote type 2 immune responses. Front Immunol 14, (2023).
33. Reid, K. T. et al. Cell therapy with IL-10-producing group 2 innate lymphoid cells suppresses Graft-versus-Host disease. bioRxiv 2023.08.21.554158 (2023) doi:10.1101/2023.08.21.554158.
34. Li, Z. et al., Therapeutic application of human type 2 innate lymphoid cells via induction of granzyme B-mediated tumor cell death, Cell, Volume 187, Issue 3 (2024), 624-641.e23.
35. Huang Q, et al., IL-10 producing type 2 innate lymphoid cells prolong islet allograft survival, EMBO Mol Med (2020) November 6; 12(11):e12305.

1. An innate lymphoid cell (ILC) expressing a chimeric antigen receptor (CAR), the CAR comprising an antigen recognition domain for recognizing a target organ or target cell, a transmembrane domain, and a signalling domain for activating signalling within the ILC.

2. The CAR-expressing ILC of claim 1, wherein the signalling domain comprises ST2(IL1RL1), CD28, CD122(IL2RB), CD132(IL2RG), CD127(IL7R), IL17RB(IL17RB), GITR (TNFRSF18), ICOS(ICOS), TNFR2(TNFRSF1B), 41BB(TNFRSF9), OX40(TNFRSF4), or CD3ζ(CD247) or a functional fragment thereof.

3. The CAR-expressing ILC of claim 2, wherein the signalling domain comprises ST2 or a functional fragment thereof.

4. The CAR-expressing ILC of claim 1, wherein the CAR further comprising a co-stimulatory domain.

5. The CAR-expressing ILC of claim 4, wherein the CAR co-stimulatory domain comprises CD28, GITR, ICOS, TNFR2, 41BB, OX40, or a functional fragment thereof.

6. The CAR-expressing ILC of claim 1, wherein the target organ or target cell is cancerous.

7. The CAR-expressing ILC of claim 1, wherein the target organ or target cell is associated with transplantation of organs or cells.

8. The CAR-expressing ILC of claim 7, wherein the target organ or target cell is associated with hematopoietic stem cell transplantation.

9. The CAR-expressing ILC of claim 7, wherein the target cell is donor derived T cells.

10. The CAR-expressing ILC of claim 7, wherein the target cell is recipient derived T cells.

11. The CAR-expressing ILC of claim 1, wherein the ILC is group 2 innate lymphoid cell (ILC2).

12. The CAR-expressing ILC of claim 11, wherein the ILC2 is IL-10 producing.

13. The CAR-expressing ILC of claim 11, wherein the ILC2 exhibits increased IL-10 expression as compared to the ILC2 prior to CAR expression.

14. The CAR-expressing ILC of claim 1, wherein the ILC is a group 3 innate lymphoid cell (ILC3).

15. The CAR-expressing ILC of claim 1, wherein the ILC is a group 1 innate lymphoid cell (ILC1).

16. A method of treating or preventing in a subject, cancer, an autoimmune disease, graft-versus-host disease, an immunodeficiency disease or an inflammatory disease, the method comprising administering to the subject the CAR-expressing ILC of claim 1.

17. A chimeric antigen receptor as defined in claim 1.

18. A nucleic acid encoding the chimeric antigen receptor of claim 17.

19. A vector comprising the nucleic acid of claim 18.

20. The vector of claim 19 being a viral vector.

21. A chimeric antigen receptor (CAR), the CAR comprising an antigen recognition domain for recognizing a target organ or target cell, a transmembrane domain, and an ST2 signalling domain.

22. A nucleic acid encoding the chimeric antigen receptor of claim 21.

23. A vector comprising the nucleic acid of claim 22.

24. The vector of claim 23 being a viral vector.

25. A cell expressing the CAR of claim 21, wherein the cell is a T cell, regulatory T cell (Treg), a myeloid cell or a macrophage, a dendritic cell (DC), a Natural Killer (NK) cell or a B cell.

26. A method for producing an innate lymphoid cell (ILC) that expresses a chimeric antigen receptor (CAR), the CAR comprising an antigen recognition domain for recognizing a target organ or target cell, a transmembrane domain, and a signalling domain for activating signalling within the ILC, the method comprising transducing the ILC with a viral vector encoding the CAR.

27. The method of claim 26, wherein the viral vector is a lentivirus vector.
